(19) United States
(12) Patent Application Publication
(10) Pub. No.: US 2008/0281169 A1
(43) Pub. Date: Nov. 13, 2008

(54) Ear-Thermometer With Ear Identification

(75) Inventors: Ton H. Akkermans, Veldhoven (NL); Daniel Schobben, Waalre (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/089,055

(22) PCT Filed: Sep. 14, 2006
(86) PCT No.: PCT/IB2006/053299

§ 371 Date: Apr. 3, 2008

Related U.S. Application Data
(60) Provisional application No. 60/725,148, filed on Oct. 7, 2005.

(51) Int. Cl. A61B 5/00; G01K 5/22

(52) U.S. Cl. 600/301; 600/549; 600/559

(57) ABSTRACT

A physiological measurement device (10) that concurrently measures a physiological state of an individual and determines an identity of the individual includes at least a measurement component (12), an identification component (14), and processing component (16). The measurement component (12) measures the physiological state of the individual, and the identification component (14) concurrently determines the identity of the individual. The processing component (16) associates the physiological state of the individual with the identity of the individual.

The following relates to sensing physiological information emitted by an individual and concurrently biometrically identifying the individual through a single device. It finds particular application to concurrently sensing a temperature of the individual and identifying the individual via an ear-thermometer with ear biometric identification componentry. Other applications are also contemplated.

Conventionally, a temperature of an individual is obtained through fluid-filled or electronic thermometers. Both types of thermometers are used to obtain oral, auxiliary, and rectal temperatures. Some electronic thermometers also measure temperature from the eardrum of the individual. For example, tympanic (“in-the-ear”) thermometers measure the temperature of the tympanum, or tympanic membrane, which is a thin membrane that separates the outer ear from the middle ear, via an infrared measurement. Typically, tympanic thermometers are simple hand-held devices with a probe that is inserted into the ear canal to measure the patient's temperature.

Electronic thermometers are commonly used at health care facilities (e.g., hospitals, clinics, doctor offices, etc.), infirmaries in businesses, educational institutions and day cares, home, etc. In these settings, a single electronic thermometer typically is used to measure the temperature of more than one individual, sometimes within a relatively short period of time. In order to prevent forgetting a temperature and/or associating a temperature with the wrong individual, the user of the electronic thermometer typically records the temperature shortly after it is obtained. By way of example, a nurse in a hospital caring for a plurality of patients typically uses the same thermometer, stethoscope, sphygmomanometer, etc. when collecting vital signs for the patients. Upon measuring the temperature of a particular patient, the nurse records the temperature, and associates the recorded temperature with the patient. For instance, the nurse may record the temperature in a patient chart or write the temperature next to a unique identification (e.g., the individual's name, social security number, patient number, room number, etc.). After obtaining and recording the patient's vital signs, the nurse replaces a disposable cover, disinfects the instrument, or the like, and moves on to the next patient. Similarly, a parent at home may have more than one ill child. Likewise, the parent typically measures, records and associates each child's temperature on a child by child basis.

Manually recording and associating temperatures of individuals is susceptible to human error. For instance, the user may inadvertently record an incorrect temperature. In another instance, the temperature of a first individual may be mistakenly associated with a different individual. In yet another instance, an identification wrist band or other identifying indicia may include erroneous information or be affixed to the wrong individual. In still another instance, the identification wrist band or other identifying indicia may have been tampered with such that the correct identity of the individual cannot be readily obtained from it. In another instance, an individual lends his/her identification wrist band to someone else, which can lead to erroneous operations, blood transfusions, etc. in the worst case leading to death. One or more of these examples as well as other situations may lead to the administration of an incorrect medication, unnecessary surgery, accidental baby swapping, etc.

A more robust approach for identifying the individual is through biometric identification such as a fingerprint, an iris map, acoustical properties of the ear, etc. By way of example, an ear probe may send a tone into the ear and listens for either reflected sound or otoacoustic emissions. Both of these approaches can be used to uniquely identify the individual through a biometric that typically cannot be lost or stolen. Even when using an ear thermometer device and a biometric ear probe or other biometric device, the user still has to manually record the temperature of the individual and associate the temperature with the correct biometrically identified individual. Thus, even when using more robust identification techniques, recorded measurements are still susceptible to human error.

In one embodiment, a physiological measurement device is illustrated. The device concurrently measures a physiological state of an individual and determines an identity the individual. The device includes a measurement component that measures the physiological state of the individual, and an identification component that concurrently determines the identity of the individual. A processing component of the device associates the physiological state of the individual with the identity of the individual.

One advantage includes concurrently measuring a physiological state of an individual and identifying the individual.

Another advantage resides in using a single device to concurrently identify an individual and measure at least one physiological state of the individual.

Another advantage lies in mitigating human error associated with manually associating physiological and identity information.

Another advantage resides in storing physiological state and identify information for a plurality of individuals.

Another advantage includes conveying correlated physiological state and identify information for one or more individuals to other components.

Still further advantages will become apparent to those of ordinary skill in the art upon reading and understanding the detailed description of the preferred embodiments.

FIG. 1 illustrates a physiological measurement system that concurrently measures physiological state information and identification of an individual.

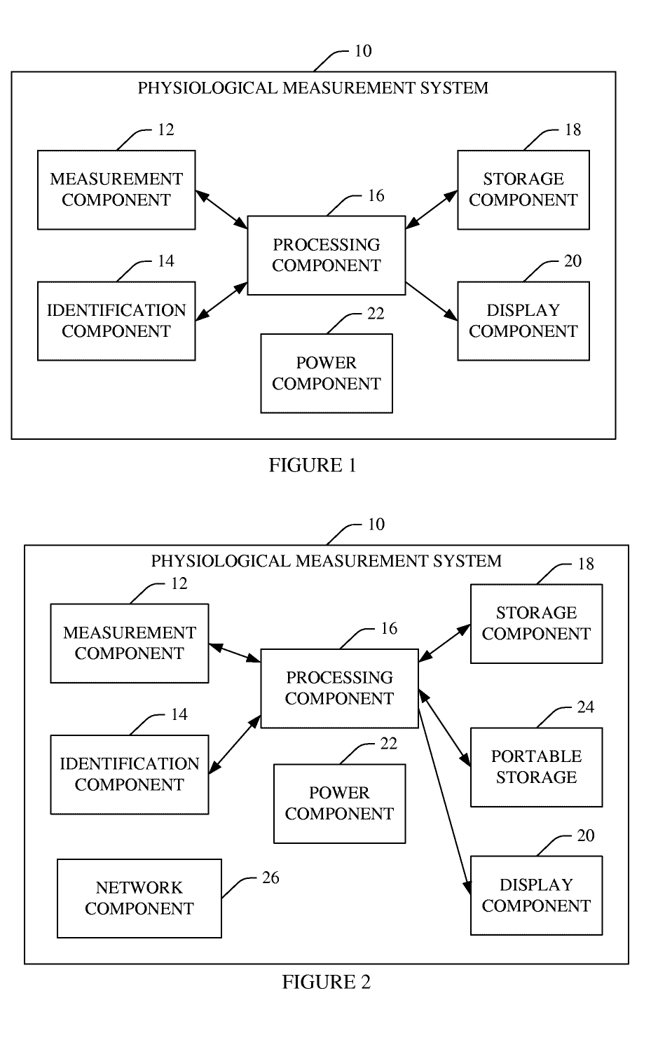

FIG. 2 illustrates another embodiment of the physiological measurement system.

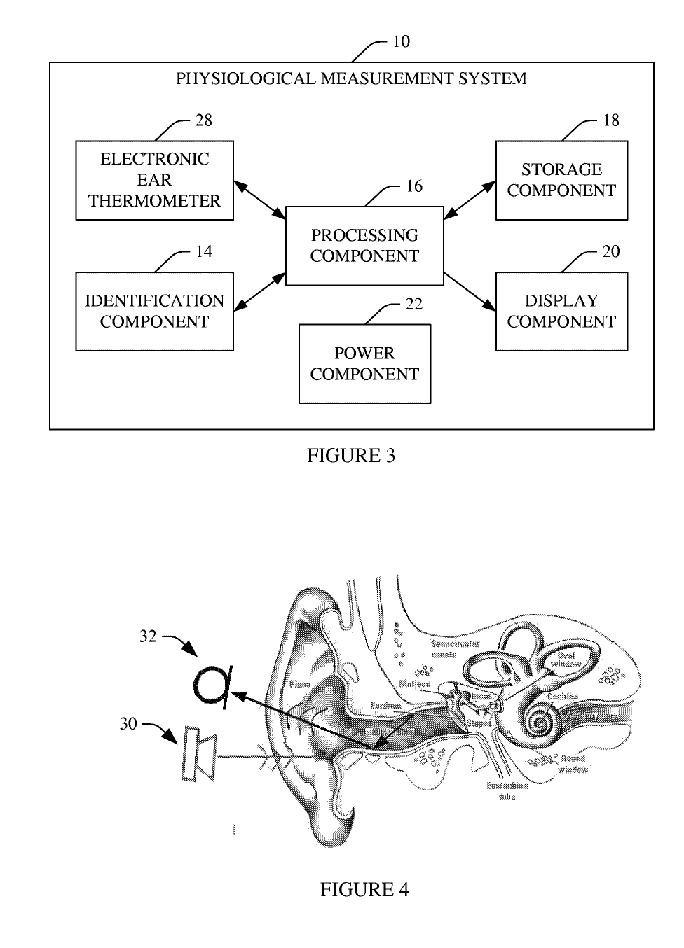

FIG. 3 illustrates an embodiment in which the physiological measurement system includes an electronic ear thermometer.

FIG. 4 illustrates a reflected sound technique for facilitating identifying an individual via ear characteristics unique to the individual.

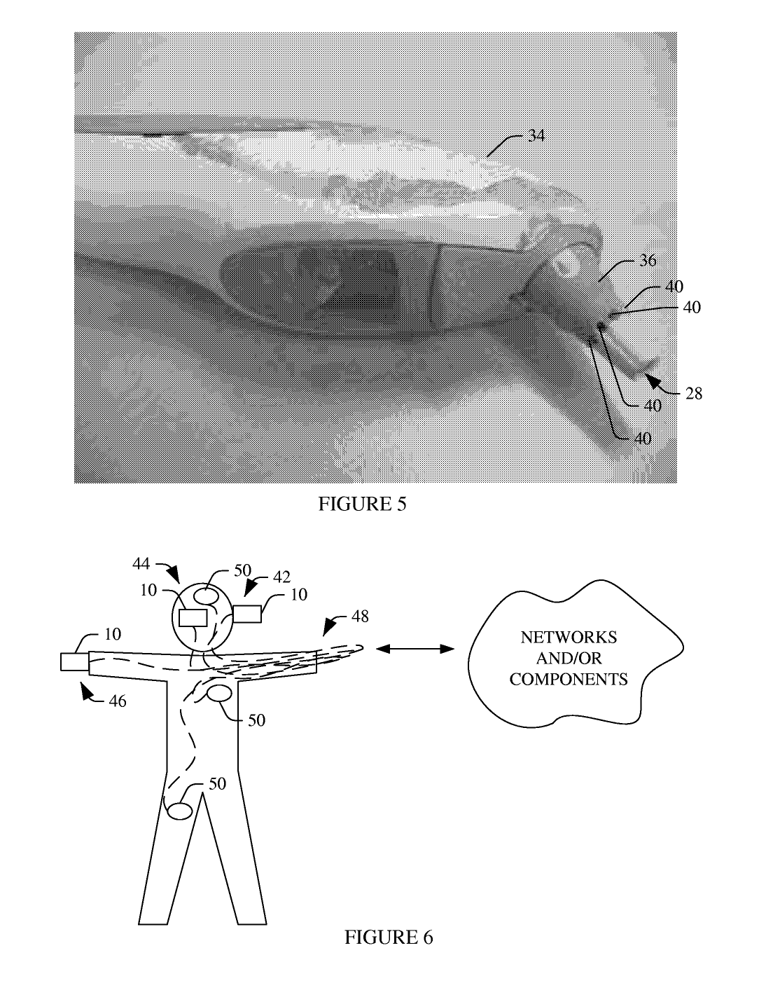

FIG. 5 illustrates a particular embodiment of the physiological measurement system having an electronic ear thermometer.

FIG. 6 illustrates the physiological measurement system within a body area network.

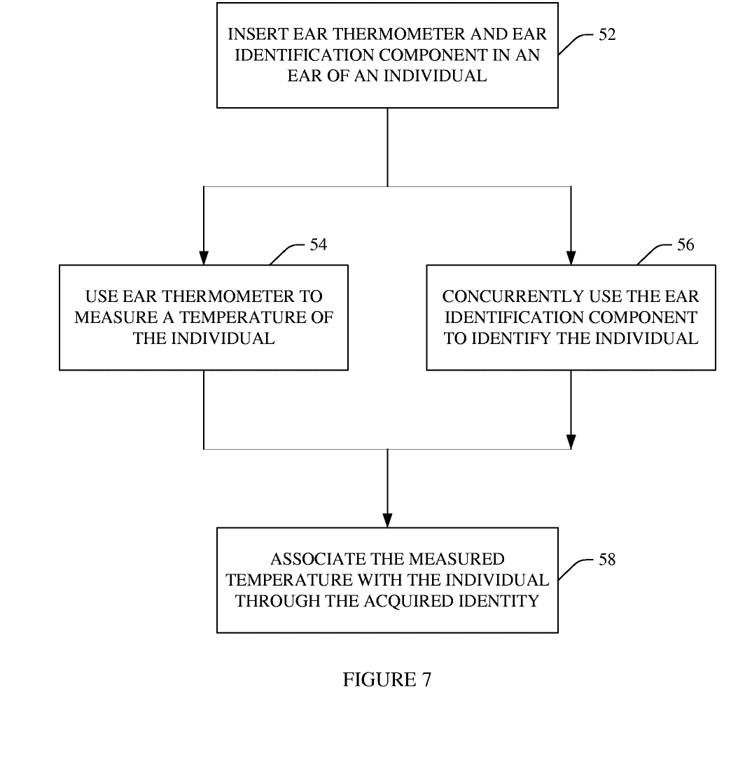

FIG. 7 illustrates a method for using the physiological measurement system to concurrently measure a temperature and identify an individual via an ear of the individual.

FIG. 1 illustrates a physiological measurement system (“system”) 10 that concurrently measures various physiological information representative of at least a physiological state and an identification of an individual. Suitable information indicative of physiological state includes, but is not limited to, temperature, pulse, blood pressure, blood-oxygen percent, heart rate, ECG/EKG or other heart electrical signals, etc., and suitable information indicative of identification includes, but is not limited to, a fingerprint, an iris map, acoustical properties of the ear, etc.

The system 10 includes a measurement component 12 for collecting physiological state information for one more physiological systems (e.g., circulatory, respiratory, cardiovascular, etc.) of the individual. For example, the measurement component 12 can include sensors for sensing temperature, blood pressure, blood-oxygen percent, heart rate, heart electrical activity, eye pressure, etc. An identification component 14 works in conjunction with the measurement component 12 in order to identify the individual while collecting the physiological state information. For example, while using a temperature sensor (not shown) of the measurement component 12 to measure a temperature from within an ear of the individual, an identification sensor (not shown) of the identification component 12 is used to identify the individual through biometrics such as unique characteristics of the ear (e.g., via reflected sound, otoacoustic (spontaneous and evoked) emissions, etc.).

In another example, while using an inner-ear blood pressure sensor (not shown) or a pulseoximeter of the measurement component 12 to measure a blood pressure or a blood oxygen level of the individual from the ear, an identification sensor (not shown) of the identification component 12 is used to identify the individual through unique characteristics of the ear. In another example, while using an eye pressure sensor (not shown) of the measurement component 12 to measure a pressure within an eye of the individual, an identification sensor (not shown) of the identification component 12 is used to identify the individual through an iris map or retinal scan. In another example, while using a finger blood pressure sensor (not shown) or a finger pulseoximeter of the measurement component 12 to measure a blood pressure or blood oxygen level of the individual, an identification sensor (not shown) of the identification component 12 is used to identify the individual through a fingerprint. It is to be appreciated that the sensor used for measuring the physiological state and for identification may be two independent sensors or substantially the same sensor. For instance, the QRS of a heart beat may be derived from information obtained from the same infrared sensor (e.g., in an ear-thermometer) used to measure temperature.

It is to be understood that the above examples are provided for explanatory purposes and are not limiting. In other embodiments, various other physiological state information is concurrently obtained along with the identity of the individual. In addition, the physiological state information and the identification may or may not be determined from substantially similar data. Furthermore, the identity information can be used to verify the identity of the individual. For instance, the identity of the individual as determined by the identification component 12 can be cross-checked with a wrist band or other mechanism or indicia used to identify the individual.

The system 10 further includes a processing component 16 that receives and associates the physiological information collected by the measurement component 12 and the identification component 14. Thus, the measured physiological information indicative of a state of one or more physiological systems of the individual is associated with the measured physiological information indicative of an identity of the individual. This association enables the system 10 to measure and store physiological state information from a plurality of different individuals. Subsequently, the physiological state information of any one of the individuals can be extracted from the stored data based on the identity of the individual.

The processing component 16 associates raw data with each individual and/or includes componentry (not shown) to process (e.g., filter, condition, amplify, threshold, etc.) the physiological state information into data more meaningful to the person acquiring the physiological information. For instance, the processing component 16 can associate resistance, current, voltage, capacitance, etc. readings with an individual and/or convert such readings to a temperature, heart rate, blood pressure, etc. and associate the processed data with the individual.

The processing component 16 stores the raw and/or processed data along with the identity of the individual in a storage component 18, which allows the user of the system 10 to measure and retain physiological information from a plurality of individuals. The storage component 18 includes volatile and/or non-volatile memory for storage of such data. The storage capacity of the storage component 18 is based on the application and cost considerations.

A display component 20 is used to present the collected physiological information to a user of the system 10. The processing component 16 presents newly acquired and/or stored physiological information on the display for the user to observe. The user determines the display information, for example, by using the system 10 to acquire physiological information and/or selecting stored physiological information. The display 20 can include at least one of one or more light emitting diodes (LEDs), seven-segment displays, liquid crystals displays, etc. Various actuators and sensors (not shown) are used to initiate measurements and/or retrieve stored data. For instance, the display 20 may include touch-screen technology to navigate through menus of options. In another instance, the system 10 may include physical buttons, dials, switches, triggers, etc. that invoke one or more actions.

A power supply 22 powers the components of the system 10. The power supply 22 can include one or more internal batteries (e.g., re-chargeable and non-rechargeable) and/or an adapter that converts AC power to suitable power.

The system 10 can be a portable device such as a hand held device or a device (removeably) affixed to a mobile structure that moves on a wheel(s), or a static device mounted to a supporting structure such as a wall or other relatively stationary instrumentation.

With reference to FIG. 2, the system 10 optionally includes portable storage 24 and/or network componentry 26. The portable storage 24 includes storage such as various types of flash memory. This enables the user of the system 10 to store and transfer physiological information by storing such information within the portable storage 24, removing the portable storage component 24 from the system 10, and connecting it to a suitable interface of another device such as a computer, a database, a monitoring station, etc. The portable storage 24 is also used to load applications, firmware, utilities, etc. onto the system 10, for example, by first storing applications, firmware, utilities, etc. on the portable storage 24, connecting the portable storage 24 to the system 10, and manually and/or automatically loading the applications, firmware, utilities, etc.

The network component 26 is also used to convey measured and/or stored physiological information to other systems. The network component 26 is associated with various wired and/or wireless communication protocols for communicating with networks such as a cellular network, a Personal Communication Service (PCS), a Wide Area Network (WAN) (e.g., the Internet), a Local Area Networks (LAN), a Metropolitan Area Network (MAN), a Campus Area Network (CAN), a Home Area Network (HAN), a Personal Area Networks (PAN), a Body Area Network (BAN), and the like. Suitable communication ports include Ethernet, Universal Serial Bus (USB), parallel, serial, optical, infrared, FireWire, etc.

Stored physiological information obtained from the system 10 via the portable storage 24 and/or the network component 26 can be analyzed and displayed according to each individual since each measurement is associated with an identity of an individual. Other information such as date, an identity of the person taking the measurement, an identity of a doctor, etc. can also be associated with the temperatures. This enables temperatures to be delineated by individual and analyzed over time, separated across the people using the system 10 to take measurements, grouped by physician, etc.

FIG. 3 illustrates a preferred embodiment in which the measurement component 12 includes an electronic ear thermometer 28. In one instance, the electronic ear thermometer 28 is a thermoelectric thermometer that uses thermoelectric current to measure temperature. This type of thermometer reads the infrared heat waves released by the eardrum. A temperature is obtained by pulling the ear backward to straighten the ear canal and aiming the ear probe between the opposite eye and earlobe. The electronic ear thermometer 28 measures the infrared heat of the eardrum and converts it to an electric signal representative of the temperature. This temperature correlates to the temperature of the hypothalamus, which is the temperature controlling system of the brain. Typically, this type of thermometer measures temperature relatively quickly (e.g., less than 2 seconds) with respect to non-ear thermometers. It also does not require cooperation by the individual (like holding a thermometer under an arm, in a mouth, etc.) and does not usually cause any discomfort.

As the electronic ear thermometer 28 measures temperature, the identification component 14 concurrently identifies the person via any suitable ear identification technique. For instance, in one embodiment a reflected sound technique is used. This embodiment is illustrated in FIG. 4 in which the identification component 14 includes a first transducer 30 (e.g., a speaker) that is used to emit a sound wave, or an excitation signal, into the eardrum and a second transducer 32 (e.g., a microphone) that captures a reflected sound wave, or echo signal. The echo signal is characteristic of the eardrum/ear canal combination and is unique to the individual.

In another embodiment, an otoacoustic approach is used. Otoacoustic emissions are weak emissions from the inner ear which can be recorded in the occluded ear canal using a relatively sensitive microphone. The otoacoustic emissions can arise spontaneously or be evoked by presenting acoustic stimuli. Applying two pure tones simultaneously in an inharmonic ratio leads to additional tones (e.g., distortion products) caused by the nonlinear behavior of the human auditory system. It is to be appreciated that the foregoing examples are provided for illustrative purpose and do not limit the invention. Other techniques such as ultrasound and the like are also contemplated.

FIG. 5 illustrates a particular example of the system 10 in which the measurement component 12 includes the electronic ear thermometer 28. Depicted is a hand-held unit having a housing 34 that protects various componentry (e.g., electronics) of the system 10 from contamination, destruction, etc. The housing 34 may be hermetically sealed to prevent ingress of fluids and/or particulate matter.

A shaft (or probe) 36 houses the ear thermometer 28. The shaft 36 is suitably designed for penetrating an opening into the ear canal. The shaft 36 is placed in the ear canal of the individual such that the ear thermometer 28 is suitably positioned for obtaining measurements. The ear thermometer 28 includes a sensor (not shown) such as an infrared sensor which collects signals emitted from with the ear canal. A transducer (not shown) of the ear thermometer 28 converts the collected signals to suitable representations such as electric currents, voltages, resistance, etc.

The shaft 36 also includes a plurality of transceivers 40 that emit acoustic signals into the ear canal and that receive signals reflected from the physical structured within the ear. The received signals are correlated with the emitted signals to obtain the physical structure in the ear and are unique to each individual such that they can be used to identify each individual. An optional sheath (not shown) can be placed over the end of the shaft 36 to protect the shaft and subsequent individuals from contamination. The sheath and/or shaft 36 can be cleanable and/or sterilizable or disposable. The temperature and the ear-identification information are concurrently obtained and associated.

FIG. 6 illustrates the system 10 residing within a wireless body area network (BAN). The system 10 is depicted at various locations proximate an individual for concurrently measuring physiological state information and identity. At a first position 42, the system 10 is positioned proximate to an ear of the individual for concurrently measuring a temperature of the individual and uniquely identifying the individual from ear characteristics. In other embodiments, blood pressure, blood oxygen level, etc. can also be acquired at 42 along with the unique identity. At a second position 44, the system 10 is positioned proximate to an eye of the individual for concurrently measuring an eye pressure, etc. of the individual and uniquely identifying the individual via an iris map. At an Nth position 46, the system 10 is positioned proximate to a finger of the individual for concurrently measuring a pulse, blood pressure, blood oxygen level, etc. of the individual and uniquely identifying the individual via a fingerprint. It is to be appreciated that these examples are provided for explanatory purposes and are not limiting. The system 10 can be used to measure various other physiological state information and identify the individual through other biometrics.

The physiological state information and identity of the individual can be displayed by the system 10 via the display component 20 and/or conveyed to other components via the portable memory 24 and/or the network componentry 26 as described in detail above. In addition, the system 10 can leverage a body area network (BAN) 48 to convey the physiological state and identity information to other components and/or networks.

One or more sensors and/or emitters 50 are also suitably placed on the individual to acquire physiological information. The one or more sensors and/or emitters 50 capture Electrocardiograms (ECGs), Electroencephalograms (EEGs), Electromyograms (EMGs), non-invasive blood pressure (NiBP), pulse, respirations, blood oxygen (SpO2), etc. In addition, the one or more sensors and/or emitters 50 can transmit information such as current medications, scheduled procedures, etc. The network component 26 can convey the associated physiological measurement and the associated patient identification of the present patient, or potentially other patients, to a corresponding receiving element of the body area network. These measurements and associated identifications are transmitted or otherwise communicated with the other data from the body area network.

FIG. 7 illustrates a method for using the physiological measurement system to concurrently measure temperature and identify an individual via an ear of the individual. At 52, a shaft that houses an ear thermometer measuring device and ear identification device is suitably positioned in the ear canal of the individual. Prior to insertion, an optional sheath can be placed over the end of the shaft to protect the shaft and individuals from contamination.

At 54, the ear thermometer measures a temperature of the individual. In one embodiment, the ear thermometer includes thermoelectric componentry that uses thermoelectric current to measure temperature by reading infrared heat waves released by the eardrum. A converter converts the measured heat waves into electric signals representative of temperature. In other embodiments, other techniques can be used to measure the temperature.

Concurrently, at 56, the ear identification device identifies the individual through unique characteristics of the ear. In one embodiment, a reflected sound technique is used in which a sound wave is emitted into the ear and a reflected sound wave is captured. The reflected sound wave is characteristic of the eardrum and is unique to the individual. In another embodiment, an otoacoustic approach is used in which a spontaneous or evoked otoacoustic emission generated via a number of different cellular mechanisms within the inner ear is captured. Similarly, this emission is characteristic of the eardrum and is unique to the individual.

At 58, the temperature and identification of the individual is associated. The temperature and identification of the individual can be displayed by the person taking the measurements. Optionally, the temperature and identification of the individual can be locally stored and/or conveyed to other components through network componentry, portable storage, and/or a body area network.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A physiological measurement device that concurrently measures a physiological state of an individual and determines an identity of the individual, comprising:
a measurement component that measures the physiological state of the individual;
an identification component that concurrently determines the identity of the individual; and
a processing component that associates the physiological state of the individual with the identity of the individual.

2. The physiological measurement device as set forth in claim 1, wherein the measurement component includes an electronic ear thermometer that measures a temperature of the individual from an ear of the individual.

3. The physiological measurement device as set forth in claim 1, wherein the identification component determines the identity of the individual from the same information used by the measurement component to determine the physiological state of the individual.

4. The physiological measurement device as set forth in claim 1, wherein the identification component uses one of a reflected sound and an otoacoustic technique to determine the identity of the individual.

5. The physiological measurement device as set forth in claim 1, wherein the measurement component measures at least one of temperature, blood pressure, eye pressure, heart rate, heart electrical activity, pulse, and blood oxygen level of the individual.

6. The physiological measurement device as set forth in claim 1, wherein the identification component determines the identity of the individual through biometric identification including at least one of a unique physical characteristic of an ear, a fingerprint, and an iris map.

7. The physiological measurement device as set forth in claim 1, further including:
a storage component for storing multiple temperatures for a plurality of individuals, wherein each temperature is associated with a corresponding individual based on the identity of the individual as determined by the identification component.

8. The physiological measurement device as set forth in claim 1, further including:
a display component for displaying the temperature and the identity of the individual.

9. The physiological measurement device as set forth in claim 1, further including:
portable storage for transferring stored temperatures and identity information from the system to devices that reads the portable storage.

10. The physiological measurement device as set forth in claim 1, further including:
a network component for conveying the temperature and the identity of the individual to other devices over a network.

11. The physiological measurement device as set forth in claim 1, further including:
a shaft that houses the measurement component and the identification component, the shaft is designed to be inserted within an ear of the individual.

12. The physiological measurement device as set forth in claim 1, the physiological measurement system uses a local body area network to convey the physiological state information and the identity of the individual to other components.

13. An ear thermometer with ear identification device, comprising:
a probe including:
an ear thermometer that measures a temperature of an individual, and
an identification component that determines an identity of the individual, the identification component;
a processing component that associates the temperature with the identity; and
a display component for presenting the temperature and an indication of the identity of the individual.

14. The ear thermometer with ear identification device as set forth in claim 13, wherein the ear thermometer includes:
a sensor that captures signals emitted from within an ear canal; and
a signal processing system that converts the collected signal to a temperature.

15. The ear thermometer with ear identification device as set forth in claim 13, wherein the sensor is an infrared sensor that captures heat waves emitted from within an ear of the individual.

16. The ear thermometer with ear identification device as set forth in claim 13, wherein the identification component includes a plurality of transceivers that emit acoustic signals into an ear canal of the individual and that receive signals reflected from the physical structured within the ear, wherein the received signals uniquely identify the individual.

17. The ear thermometer with ear identification device as set forth in claim 13, wherein the identification component uses one of a reflected sound and an otoacoustic approach to determine the identity of the individual.

18. The ear thermometer with ear identification device as set forth in claim 13, further including at least one of:
portable storages; and
a network component for transferring the temperature and the identity of the individual to other devices.

19. A method for measuring a physiological state of an individual and determining an identity the individual, comprising:
concurrently determining:
a physiological state of the individual, and
an identity of the individual; and
associating the physiological state with the identity of the individual.

20. The method as set forth in claim 19, wherein the physiological state is one of a temperature, a blood pressure, an eye pressure, a heart rate, a heart electrical signal, a pulse, and a blood oxygen level.

21. The method as set forth in claim 19, further including:
storing a plurality of temperatures, each of which is associated with an identity of a corresponding individual;
displaying the plurality of temperatures with the identity information; and
transferring the stored temperatures and identity information to at least one other device.
